(19) United States
(12) Patent Application Publication
(10) Pub. No.: US 2021/0057109 A1
(43) Pub. Date: Feb. 25, 2021

(54) METHOD FOR IDENTIFYING AND QUANTIFYING POPULATIONS EXPOSED TO ENVIRONMENTAL HAZARDS ACROSS A GEOGRAPHIC REGION

(71) Applicants: Peter Keith Rogan, London (CA); Eliseos J. Mucaki, London (CA)

(72) Inventors: Peter Keith Rogan, London (CA); Eliseos J. Mucaki, London (CA)

(21) Appl. No.: 16/996,792

(22) Filed: Aug. 18, 2020

Related U.S. Application Data
(60) Provisional application No. 62/889,090, filed on Aug. 20, 2019.
(60) Provisional application No. 63/007,672, filed on Apr. 9, 2020.

(51) Int. Cl. G16H 50/80; G06Q 30/02

(52) U.S. Cl. CPC G16H 50/80; G06Q 30/0205

(57) ABSTRACT

The present invention discloses a method for identifying and quantifying populations exposed to environmental hazards across a geographic region. The environmental hazards include radiation, pollution and communicable infectious agent hotspots, such as locations of COVID-19 hotspots. The method of the present invention uses geographic distributions of infected individuals over time to develop robust methods that pinpoint locations of emerging COVID-19 hotspots. The method assays a fraction of infected individuals of a local population and adjacent locations of the infected individuals and detects spatial asymmetries and clustered distributions of infected individuals. The spatial resolution of the assay is increased by assigning infected cases in each county to subdivisions weighted by population census and performing spatial interpolation to pinpoint potential local clusters of infected individuals.

### CROSS-REFERENCES TO RELATED APPLICATIONS

This nonprovisional application claims benefit of the filing dates of pending provisional applications 62/889,090 filed on 2019 Aug. 20 and 63/007,672 which was filed on 2020 Apr. 9. The contents of application nos. 62/889,090 and 63/007,672 are hereby incorporated by reference in their entirety.

### TECHNICAL FIELD

The present disclosure relates generally to geostatistical analytic techniques and environmental hazards, and more particularly, to a method for identifying and quantifying populations exposed to environmental hazards across a geographic region utilizing geostatistical analytic techniques.

### OTHER CROSS-REFERENCES

The following articles are hereby incorporated by reference in their entirety.
Peter K. Rogan, Eliseos Mucaki, Ruipeng Lu, Ben C. Shirley, Edward Waller and Joan H. M. Knoll. Meeting radiation dosimetry capacity requirements of population-scale exposures by geostatistical sampling; Short Title Reducing radiation dosimetry testing by geostatistical sampling. medRxiv https://doi.org/10.1101/2020.04.08.20058446 (Apr. 14, 2020).
Peter K. Rogan, Eliseos Mucaki, Ruipeng Lu, Ben C. Shirley, Edward Waller and Joan H. M. Knoll. Meeting radiation dosimetry capacity requirements of population-scale exposures by geostatistical sampling. PloS ONE 15(4): e0232008 (Apr. 24, 2020). https://doi.org/10.1371/journal.pone.0232008.
McGrory K. Coronavirus may have caused hundreds of additional deaths in Florida, Tampa Bay Times, May 20, 2020.
Peter K Rogan. (2020). Geostatistical Analysis of SARS-CoV-2 in the United States (Version 1) [Data set]. Zenodo. http://doi.org/10.5281/zenodo.3890285

### BACKGROUND

Geostatistics is used to analyze and predict the values associated with spatial or spatiotemporal phenomena. It incorporates the spatial and/or temporal coordinates of the data within the analyses. Geostatistics provides a powerful tool to describe spatial patterns and interpolate values for locations and also measures uncertainty for those values. The measurement of uncertainty is critical for decision making, as it provides information on the possible values or outcomes for each location rather than just one interpolated value.

The geostatistical analysis uses regression or kriging methods to interpolate environmental measurements across a range of spatial coordinates. Kriging interpolates the value of unsampled locations by computing weight linear estimates at these locations using neighbouring data. The mining industry applications of kriging to estimate the contiguous distribution of mineral deposits from a limited number of samples motivated the study to apply kriging for geographic extrapolation of absorbed radiation from a fraction of potentially exposed individuals. This would help in the prediction of environmental contaminant levels and their relation to the incidence rates of diseases such as cancer or other environmental hazards, communicable infectious agents such as COVID-19 hotspots.

Accordingly, a method for quantifying individual exposures in a population of individuals across a geographic region to environmental hazards utilizing geostatistical analysis is needed.

### SUMMARY

The present invention discloses a method for identifying and quantifying populations exposed to environmental hazards across a geographic region utilizing geostatistical analytic techniques. The environmental hazards include, but not limited to, radiation, pollution, and communicable infectious agents. The methods described herein include steps requiring a computing machine such as a computer having a processor, monitor, and data storage media.

In one embodiment, a method for identifying and quantifying populations exposed to environmental hazards across a geographic region is disclosed. At one step, data related to the source of the hazard is provided as input. In one embodiment, the hazard is a communicable infectious agent. In one embodiment, the data includes the location of individuals infected by the infectious agents, the number of infected individuals and the location of the infected individuals. At another step, a fraction of infected individuals of a local population and adjacent locations of the infected individuals are assayed. At yet another step, spatial asymmetries and clustered distributions of infected individuals are detected. At yet another step, the spatial resolution of the assay is increased by assigning infected cases in each county to subdivisions weighted by population census, and spatial interpolation is performed to pinpoint potential local clusters of infected individuals. At yet another step, integrated risk scores are assigned to potential clusters and a geographic map annotated with potential local clusters of infected individuals is computed.

In one embodiment, a method for quantifying individual biological exposures to ionizing radiation in a population of individuals is disclosed. At one step, the target location, wind direction and speed of the radiation source are provided as input. At another step, a fraction of localized individuals of the local population that may have been exposed to the radiation is sampled. In one embodiment, the fraction comprises at least 0.01% of the local population count at the target location and adjacent locations dictated by the wind direction. At yet another step, the absorbed biological radiation exposure level of sampled individuals by biodosimetry is determined. At yet another step, a geographic map of the biological distribution of radiation exposures of all individuals proximate to the radiation source is computed using geostatistical methods that spatially infer radiation exposure contours from the absorbed radiation level and the location of each sampled individual. In one embodiment, the test includes cytogenetic, gene and protein expression and metabolomic signatures, and electron paramagnetic resonance biodosimetry. In one embodiment, the radiation levels are inferred by either ordinary, simple, universal, or empirical Bayesian kriging or other non-linear regression approaches.

In one embodiment, a densification method of improving the quantification of individual biological exposures to ionizing radiation in a population of individuals is disclosed. At one step, locations on the biodosimetry geographic map with the highest levels of uncertainty in radiation dose estimates are determined. At another step, additional individuals at or close to the locations are sampled and their respective biological radiation exposure levels are determined. At yet another step, a geographic map of the biological distribution of radiation exposures of all individuals proximate to the radiation source is computed using geostatistical methods that spatially infer radiation exposure contours from the absorbed radiation level and the location of each sampled individual, including the additional individuals sampled.

In one embodiment, the quantification method could be improved by repeating the cycles of kriging and densification using the biodosimetry geographic map as input for densification and geostatistical inference of the improved map. The convergence of the improved quantification method could be determined by comparing overlapping grids between each pair of contours of successive plumes based on minimizing differences between either their corresponding diagonal Bray-Curtis distances or their root-mean-square deviations.

In one embodiment, a method of quantifying individual physical exposures to ionizing radiation in a population of individuals is disclosed. At one step, the target location, wind direction and speed of the radiation source are provided as input. At another step, physical dosimeters are placed at the locations of a fraction of individuals of a local population which is suspected of having been exposed to radiation. In one embodiment, the fraction comprises at least 0.01% of the local population count, relative to the location of the target of the radiation event and adjacent locations dictated by the wind direction. At another step, the radiation exposure level measured by the dosimeters are determined. At yet another step, a geographic map of the distribution of radiation exposures of all individuals proximate to the radiation source is computed using geostatistical methods that spatially infer radiation exposure contours from the absorbed radiation level and each sampled location. In one embodiment, the testing equipment includes Geiger Mueller detectors with pancake probes, alpha radiation survey meters, dose rate meters, personal dosimeters, and portal monitors. In one embodiment, the radiation levels are inferred by either ordinary, simple, universal, or empirical Bayesian kriging or other non-linear regression approaches.

In one embodiment, a densification method for improving the quantification of radiation exposure across a geographic region to radiation exposing a population of individuals is disclosed. At one step, locations on the geographic map with the highest levels of uncertainty in radiation dose estimates are determined. At another step, the additional dosimeters at or close to those locations are sampled and their respective radiation exposure levels are determined. At another step, a geographic map of the distribution of physical radiation exposures of all individuals proximate to the radiation source is recomputed using geostatistical methods that spatially infer radiation exposure contours from the measured radiation levels and the location of each sampled location, including the additional dosimeter measurements. In one embodiment, the quantification of individual physical exposures to ionizing radiation in a population of individuals could be improved by repeating the densification method using the dosimetry geographic map as input for densification and geostatistical inference of the improved map. The convergence of the improved quantification can be determined by comparing overlapping grids between each pair of contours of successive plumes based on minimizing differences between either their corresponding diagonal Bray-Curtis distances or root-mean-square deviations.

In one embodiment, a method of quantifying individual exposes in a population of individuals across a geographic region to environmental hazards are disclosed. In one embodiment, the hazards, includes, but not limited to, radiation, pollution and communicable infectious agents. At one step, the target location, direction and rate of dispersal of the source of the hazard are provided as input. At another step, dose at the locations of a fraction of individuals of a local population which is suspected of having been exposed to radiation are assayed. In one embodiment, the fraction comprises at least 0.01% of the local population count, relative to the location of the target of the event and adjacent locations dictated by the direction of travel of the hazard. At yet another step, the exposure level measured by assaying the environmental hazard at these locations is determined. At yet another step, a geographic map of the distribution of exposures to the environmental hazard of all individuals proximate to the radiation source is computed using geostatistical methods that spatially infer exposure contours from the level of the environmental hazard and each sampled location.

In one embodiment, a densification method for quantifying exposures to a population of individuals across a geographic region to environmental hazards. In one embodiment, the hazards include radiation, pollution and communicable infectious agents. At one step, locations on the geographic map with the highest levels of uncertainty in radiation dose estimates are determined. At another step, additional environmental hazard assays at or close to those locations are sampled and their respective exposures are determined. At yet another step, a geographic map of the distribution of physical radiation exposures of all individuals proximate to the radiation source is recomputed using geostatistical methods that spatially infer radiation exposure contours from the measured radiation levels and the location of each sampled location, including the additional measurements. At yet another step, a new set data on the quantifying exposures to a population of individuals across a geographic region to environmental hazards are obtained sequentially. At yet another step, locations on the geographic map with the highest levels of uncertainty in radiation dose estimates is determined. At yet another step, coincident between any of the location obtained by densification. At yet another step, the coincident location is assigned as the emerging hotspots for increased exposure to environmental hazards.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 exemplarily illustrates a method for high-level daily processing of COVID-19 counts, according to an embodiment of the present invention.

FIG. 2 exemplarily illustrates a screenshot of a map indicating Louisiana COVID-19 hotspots, according to an embodiment of the present invention.

FIG. 3 exemplarily illustrates a screenshot of a map indicating the expanded hotspot of parish centroid locations from the empirical bayesian kriging method, according to an embodiment of the present invention.

FIG. 4 is a screenshot illustrating the densification of spatially interpolated hotspots, according to an embodiment of the present invention.

FIG. 5 exemplarily illustrates a flowchart of a method for quantifying individual biological exposures to ionizing radiation in a population of individuals, according to an embodiment of the present invention.

FIG. 6A and FIG. 6B exemplarily illustrates predicted radiation levels based on the distribution of dose estimated patients around the prediction location, according to an embodiment of the present invention.

FIG. 7 exemplarily illustrates a flowchart of a method for quantifying individual biological exposures to ionizing radiation in a population of individuals, according to another embodiment of the present invention.

### DETAILED DESCRIPTION OF EXAMPLE EMBODIMENTS

The present invention discloses a method for identifying and quantifying populations exposed to environmental hazards across a geographic region. The environmental hazards includes, but not limited to, radiation, pollution, and communicable infectious agents such as corona virus.

The method of the present invention uses geographic distributions of infected individuals over time to develop robust methods that pinpoint locations of COVID-19 hotspots, which are likely sentinels for the spread of SARS-CoV-2 infections. In one embodiment, the method utilizes publicly available daily county-level COVID-19 epidemiology data, location intelligence, and map visualization technology. The method applies and develops geostatistical spatial and spatiotemporal tests based on this data to identify emerging COVID-19 hotspots across the geographic location. The method further uses colocalization and expansion of clusters of infected individuals over time for early identification of emerging COVID-19 hotspots.

The method of the present invention provides rigorous, hypothesis-based testing of trends and relationships between spatial locations over time of COVID-19 cases. These patterns are often not obvious from the daily stochastic changes in count data, and potentially identifies hotspots earlier than seen in cumulative count distributions. The method also provides a spatial interpolation approach to pinpoint clusters of COVID-19 infected individuals within county subdivisions by allocating county-level case counts among at random locations within subdivisions, according to the respective populations of each subdivision. This could facilitate effective contact tracing and mitigation strategies to be focused and deployed at these locations, thereby monitoring efforts to halt community spread.

The method enables early identification of COVID-19 hotspots through examination of differences in the regional spatial and temporal distributions of infected individuals. Geostatistical analysis of infected individuals focuses efforts to identify locations proximate to sources of transmission. Using retrospective incidence data, the method applies established geostatistical tests for spatial asymmetry, clustering and hotspot analysis for early identification of counties where cases have accumulated more rapidly than surrounding locations. The method facilitates localizing of hotspots beyond county-level resolution and integrates spatiotemporal results as risk scores to inform management of potential hotspots.

The method enables spatial interpolation by kriging at higher granularity that currently available from public health sources. Densify sampling procedures are then used to estimate the population-weighted average locations of COVID-19 hotspots. This procedure identifies sampling locations after kriging to a monitoring network. The selection criteria are used to determine where to sample, including the maximum prediction uncertainty and the highest probability that a specified threshold value of COVID-19 counts is exceeded. Based on maximum prediction standard error across locations, stderr(s), as the criterion for location selection, densification chooses a new location that minimizes stderr(s). The optimality criterion O0(s) is expressed as the maximum of stderr(s) over the interpolated region. In practice, the interpolated hotspot location is expected to exhibit increased counts relative to surrounding areas. The probability of exceeding a count threshold is used to weigh the prediction standard error or the interquartile range (interquartile range Z0.75(s)-Z0.25(s) is often used rather than stderr(s) if it is distributed across s asymmetrically): O1(s)=maximum of stderr(s)(1−2·abs(prob[Z(s)>Zthreshold]−0.5)). The criterion value decreases as the uncertainty about exceeding the threshold value decreases. Adding the locations with the largest weighted prediction standard error O0(s) to the monitoring network will improve predictions near the threshold value.

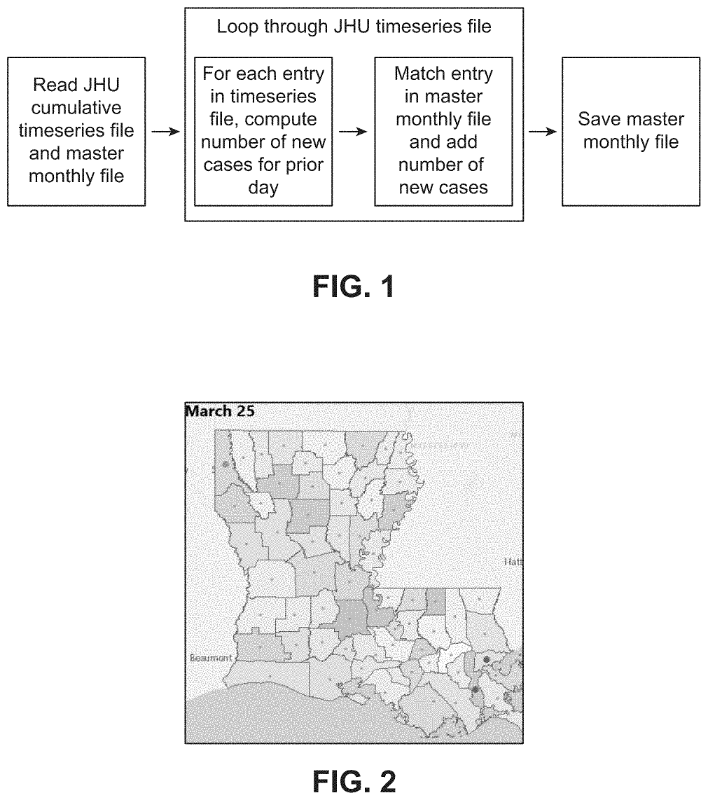

FIG. 1 exemplarily illustrates a method for high-level daily processing of COVID-19 counts, according to an embodiment of the present invention. Initially, county-level incidence data is collected from public repository, for example, Johns Hopkins University Coronavirus Github repository. The method programmatically converts cumulative count values to newly ascertained cases, classifying these results and linking each entry county centroid locations associated with specific subsets.

The statistical tests are used to assess data quality based on identification of missing values, counts not attributed to specific locations, corrections of prior day time series resulting in negative values, and identification of unlikely probabilistic outliers. The correction of negative values involves subtraction of the values from the preceding day and assign zero cases to the day with the negative count. However, large value corrections would require recursive propagation of this process until no further negative cases occur, which may not be accurate. Alternatively, reallocation of counts could be avoided by reassigning all negative case counts to zero. This issue highlights the importance of trend (spatial and temporal) analysis that does not rely on a single day count values in estimating potential hotspots. The technical glitches could also lead to revision of counts and their classification. For example, an isolated one-day spike of cases with unknown geographic origin in Michigan was the result of an unannounced change in the hosting URL that affected the performance of the software that populates new entries in the Github repository. While these anomalies could be detected statistically, procedures for correction remain ambiguous. Thus, anomalies would be tagged on a dedicated map layer in the webserver annotating results by location.

In one embodiment, the systemic undercounting of COVID-19 cases addressed by correction of overall count data in each state. Annual mortality statistics of unspecified pneumonia would be compared between the same time period in 2020 vs prior years for possible unrecognized COVID-19 cases, which has been suggested to be likely between 17 and 58% higher than published mortality rates. To address underreporting, the populations of each county would be used to weight the missed case counts among individual counties and added to daily totals. There is often a delay in reporting mortality statistics, which would limit the application of this approach for all retrospectively obtained COVID-19 data. Uncorrected and mortality corrected COVID-19 results would be compared to determine whether undercounting affects area-to-area spatial asymmetry, clustering statistics, hotspot determination, or the results of point-to-point estimation of counts based on kriging and densification.

Further, the method of the present invention analyze data retrospectively across the geographic region and apply methods that have been shown to detect spatial asymmetries and clustering, and emerging hotspots. These approaches identify potential geographic disparities between adjacent counties using area-to-area geostatistical analysis. In one embodiment, methods such as Global Moran's I8 and Anselin Local Moran's I9 test are applied for spatial asymmetric and clustered distributions of affected cases. This could reveal clusters of counties above background COVID-19 levels as well as mixed regions where either high or low infection levels predominate. These tests generate statistics (Moran's Index, Variance, Z-Score, p-value) that indicate features which cluster spatially with other neighboring features. Further, the method specifically locates persistent hotspots with several approaches: 1) using Getis-Ord Gi* test based on Euclidean distance-based or K-nearest neighbor criteria for adjacent regions; 2) incorporate temporal trends when a spatial weights matrix is incorporated that provides a time barrier between data points at the same locations; and 3) an optimized analysis that locates and ignores outlier data, converts remaining input to weighted features, then scales results, applies a false discovery rate correction prior to testing; and 4) emerging hotspot analyses to identify trends within space-time NetCDF cubes which are an aggregate of points placed into space-time bins. This analysis then takes the cube as input and identifies statistically significant, monotonic hot and cold spot trends over time using the Mann-Kendall rank correlation analysis. Variance within these bins over time are compared to expected values to determine statistical significance.

Further, point-to-point spatial interpolation of COVID-19 counts as a means of effectively increasing the geographic resolution of hotspot predictions. Counts at unsampled locations are determined by kriging, i.e. from weighted linear averages at these locations using neighbouring data. Coordinates of affected individuals will be randomized within county subdivisions, with counts allocated to and weighted according to population census in respective county subdivisions. The subdivision KML boundaries and population data are obtained from the census bureau. Geostatistical analysis of count data is imported to ArcGIS using our previously customized Python scripts calling the ArcGIS software toolkit.

In one embodiment, the method evaluates hotspot predictions using Poisson and other types of kriging that have been used to reconstruct geographic incidence using disease-related count data. Kriging of county incidence values are performed assuming a uniform population distribution which are compared with counts of affected cases distributed across county subdivisions weighted by population census. In some instances, this will reveal potential hotspots derived from contiguous subdivisions in adjacent counties. In one embodiment, locations with high COVID-19 count standard errors are selected over a narrow time window of dates to determine whether spatial interpolation can more precisely define these locations of emerging hotspots compared to area-to-area approaches. The results of kriging and densification are displayed as a geostatistical layer, created using a kriging model with measurements at the existing locations, to determine prediction standard error, the interquartile range, and the probability that a specified threshold is exceeded for every input location.

The validated hotspots are more likely to exhibit increased rates in affected cases relative to surrounding areas. Significant results will be evaluated by demonstrating absence of hotspots in simulated realistic datasets, assessing both their sensitivity and specificity using ROC curves. The methods will also be validated with retrospective examples of well-known COVID hotspots. Predicted hotspots will also be searched in the media for reports at these locations to determine whether the predictions preceded public release of this information.

The method provides a composite risk score that combines the results of join point regression modelling of temporal trends and boundary analysis for each hotspot candidate. Trend lines are analysed based on variance in counts of affected individuals for the same hotspot at different times after the initial hotspot is identified. Each trend model consists of a series of linear segments connected at join points, whose values and numbers are estimated by an iterative procedure. Statistically significant changes in the trending hotspots are detected by a Monte Carlo permutation method. A grid search is used to fit the segmented regression function and the p-value of each permutation test is estimated using Monte Carlo methods, and the overall asymptotic significance level is maintained through a Bonferroni adjustment. For each county and time step, trend models for adjacent counties are compared and the percentage of neighbors with statistically different trend models are used to create a composite risk score, which supplements the outlier detection conducted using indicators of negative spatial autocorrelation.

The present invention further provides a web-based, publicly available server that indicates the geolocations of emerging hotspots of COVID-19 infections across the geographic location. The server is configured to provides map layer for area-to-area spatial asymmetry, geospatial hotspot prediction, temporal trends in COVID-19 counts, kriging results, and potential hotspots derived interpolated high variance locations derived by densification. The server is further configured to provide a layer showing the risk scores for spatiotemporally integrated hotspot results. Finally, a summary map layer of annotations that categorizes hotspots according to type and degree of hotspot verification will be created from: 1) data that have been independently verified from public media reports, 2) comparisons with simulated kriging and densification maps, 3) verified infection rate differences relative to neighboring counties, and 4) the most significant risk scores. The hotspots that do not fulfill criteria requirements for inclusion in the summary map layer will be revaluated to revise the detection criteria for these locations in subsequent analyses of COVID-19 incidence data.

### Examples

The results of dynamic spatial and temporal analyses of COVID-19 positive cases for the same tests and geographic regions applying the method of the present invention is disclosed as follows.

The 2020 Mardi Gras festival was predicted to cause an increase in COVID-19 cases. SE Louisiana: Orleans and Jefferson parishes were reported as hotspots in the media in late March. This was confirmed with the distance-based Getis-Ord Gi* test, shown in FIG. 2. Unexpectedly, Caddo parish (NW Louisiana) was also found to be a hotspot on this date. The mayor of Shreveport publicly announced this spike in cases one-week later. This gives us optimism that this approach will be useful for detecting emerging clusters after community transmission of COVID-19, possibly in time to mitigate against potential transmission to unaffected individuals.

We also generated a matrix of COVID-19 counts for Louisiana parishes over two time periods, which was then used to assign time-space based spatial weights for the above hotspot analysis. From March 24-April 4, and from March 24-April 9 using the ArcGIS default threshold distance between adjacent samples (53 km) for optimal hotspot analysis, Caddo parish remains significant, although the confidence interval is decreased in the expanded time window. Interestingly, the Iberville parish (Baton Rouge) is also indicated as a hotspot in the expanded time window.

An emerging hotspot analysis (which requires ≥10-day window) was carried out over several windows: from March 24 to either April 2, or to April 4, or to April 6, or to April 9 or to May 31, 2020. Regardless of the date range, the only region highlighted as an emerging, consecutive or persistent hot spot was over Jefferson parish. Varying the neighborhood time step over 2 days caused the hotspot region to expand, but it remains centered over the Jefferson/Orleans region.

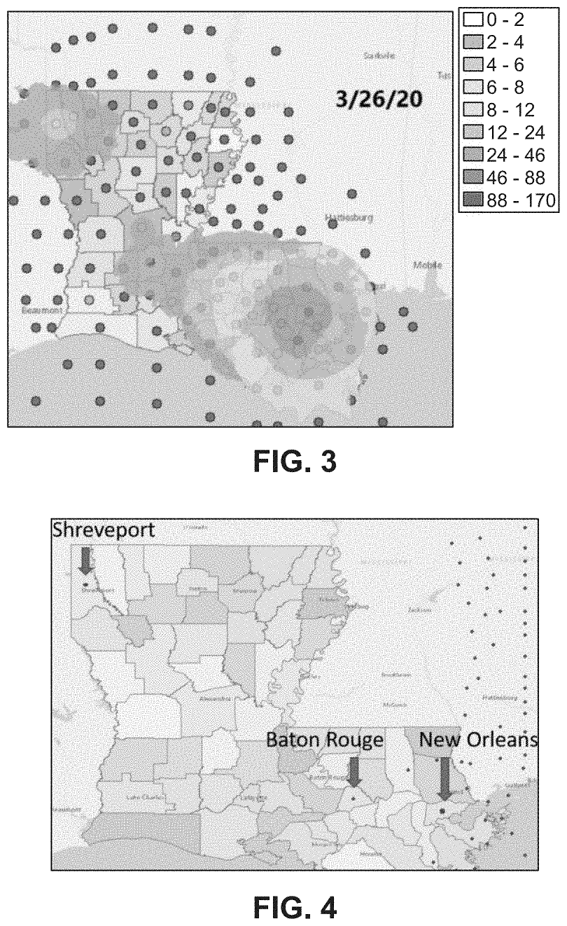

Referring to FIG. 3, the size of the expanded hotspot is evident from Empirical Bayesian Kriging of parish centroid locations. However, densification selected Caddo parish, Orleans and Iberville location as a hotspot on both March 25 and 29, as shown in FIG. 4. Thus, hotspots derived by kriging, while more precise, may not be consistently delineated as they emerge. Densification procedures weighted towards locations with counts exceeding a defined threshold are less noisy but can exclude weaker hotspots. These results indicate the potential importance of risk scoring of prospective hotspots (Aim 4) for directing contact tracing.

To validate these predictions, we performed time linear regression on the rate of COVID-19 counts for the predicted hotspots relative to the surrounding parishes. Three parishes had higher slopes (cases/day) than Caddo from Mar. 29, 2020 (East Baton Rouge [slope=+28.0], Jefferson [+84.2], Orleans [79.8]; R2˜0.5). Beginning on March 29, the slope in Caddo parish was +23.8 (R2=0.49). The parishes immediately surrounding Caddo exhibit significantly lower slopes during the March 29-April 9 window: Bossier [+2.9], De Soto [+2.4], Red River [−0.04]; with R2 ranging from 0.4-0.5, except for Red River which is uncorrelated due to lack of COVID-19 cases.

In one embodiment, a method for identifying and quantifying populations exposed to environmental hazards across a geographic region is disclosed. At one step, data related to the source of the hazard is provided as input. In one embodiment, the hazard is a communicable infectious agent. In one embodiment, the data includes location of individuals infected by the infectious agents, number of infected individuals and location of the infected individuals. At another step, a fraction of infected individuals of a local population and adjacent locations of the infected individuals is assayed. At yet another step, spatial asymmetries and clustered distributions of infected individuals are detected. At yet another step, the spatial resolution of the assay is increased by assigning infected cases in each county to subdivisions weighted by population census and spatial interpolation is performed to pinpoint potential local clusters of infected individuals. At yet another step, integrated risk scores are assigned to potential clusters and a geographic map annotated with potential local clusters of infected individuals is computed.

In one embodiment, a method for quantifying individual biological exposures to ionizing radiation in a population of individuals is disclosed. At one step, the target location, wind direction and speed of the radiation source are provided as input. At another step, a fraction of localized individuals of the local population that may have been exposed to the radiation is sampled. In one embodiment, the fraction comprises at least 0.01% of the local population count at the target location and adjacent locations dictated by the wind direction. At yet another step, the absorbed biological radiation exposure level of sampled individuals by biodosimetry is determined. At yet another step, a geographic map of the biological distribution of radiation exposures of all individuals proximate to the radiation source is computed using geostatistical methods that spatially infer radiation exposure contours from the absorbed radiation level and the location of each sampled individual. In one embodiment, the test includes cytogenetic, gene and protein expression and metabolomic signatures, and electron paramagnetic resonance biodosimetry. In one embodiment, the radiation levels are inferred by either ordinary, simple, universal, or empirical Bayesian kriging or other non-linear regression approaches.

In one embodiment, a densification method of improving the quantification of individual biological exposures to ionizing radiation in a population of individuals is disclosed. At one step, locations on the biodosimetry geographic map with the highest levels of uncertainty in radiation dose estimates is determined. At another step, additional individuals at or close to the locations are sampled and their respective biological radiation exposure levels are determined. At yet another step, a geographic map of the biological distribution of radiation exposures of all individuals proximate to the radiation source is computed using geostatistical methods that spatially infer radiation exposure contours from the absorbed radiation level and the location of each sampled individual, including the additional individuals sampled.

In one embodiment, the quantification method could be improved by repeating the densification method using the biodosimetry geographic map as input for densification and geostatistical inference of the improved map. The convergence of the improved quantification method could be determined by comparing overlapping grids between each pair of contours of successive plumes based on minimizing differences between either their corresponding diagonal Bray-Curtis distances or their root-mean-square deviations. In the instant invention, convergence was achieved using a stopping criteria of <10% difference in areas between successive iterations of results of the kriging-densification cycling. However, those of skill in the art recognize that the stringency of this stopping criteria can be increased or decreased according the the specific requirements to measure the environmental effect. For example, an immediate response would be required to apply the method in the case either unexpected, large scale nuclear radiation incident or an imminent infectious disease pandemic, and in such cases, the stopping criteria may be more relaxed (>10% difference between results of successive iterations) to provide rapid information to first responders.

In one embodiment, a method of quantifying individual physical exposures to ionizing radiation in a population of individuals is disclosed. At one step, the target location, wind direction and speed of the radiation source are provided as input. At another step, physical dosimeters are placed at the locations of a fraction of individuals of a local population which is suspected of having been exposed to radiation. In one embodiment, the fraction comprises at least 0.01% of the local population count, relative to the location of the target of the radiation event and adjacent locations dictated by the wind direction. At another step, the radiation exposure level measured by the dosimeters are determined. At yet another step, a geographic map of the distribution of radiation exposures of all individuals proximate to the radiation source is computed using geostatistical methods that spatially infer radiation exposure contours from the absorbed radiation level and each sampled location. In one embodiment, the testing equipment includes Geiger Mueller detectors with pancake probes, alpha radiation survey meters, dose rate meters, personal dosimeters, and portal monitors. In one embodiment, the radiation levels are inferred by either ordinary, simple, universal, or empirical Bayesian kriging or other non-linear regression approaches.

In one embodiment, a densification method for improving the quantification of radiation exposure across a geographic region to radiation exposing a population of individuals is disclosed. At one step, locations on the geographic map with the highest levels of uncertainty in radiation dose estimates are determined. At another step, the additional dosimeters at or close to those locations are sampled and their respective radiation exposure levels are determined. At another step, a geographic map of the distribution of physical radiation exposures of all individuals proximate to the radiation source is recomputed using geostatistical methods that spatially infer radiation exposure contours from the measured radiation levels and the location of each sampled location, including the additional dosimeter measurements. In one embodiment, the quantification of individual physical exposures to ionizing radiation in a population of individuals could be improved by repeating the densification method using the dosimetry geographic map as input for densification and geostatistical inference of the improved map. The convergence of the improved quantification can be determined by comparing overlapping grids between each pair of contours of successive plumes based on minimizing differences between either their corresponding diagonal Bray-Curtis distances or root-mean-square deviations.

In one embodiment, a method of quantifying individual exposures in a population of individuals across a geographic region to environmental hazards are disclosed. In one embodiment, the hazards, includes, but not limited to, radiation, pollution and communicable infectious agents. At one step, the target location, direction and rate of dispersal of the source of the hazard are provided as input. At another step, dose at the locations of a fraction of individuals of a local population which is suspected of having been exposed to radiation are assayed. In one embodiment, the fraction comprises at least 0.01% of the local population count, relative to the location of the target of the event and adjacent locations dictated by the direction of travel of the hazard. At yet another step, the exposure level measured by assaying the environmental hazard at these locations is determined. At yet another step, a geographic map of the distribution of exposures to the environmental hazard of all individuals proximate to the radiation source is computed using geostatistical methods that spatially infer exposure contours from the level of the environmental hazard and each sampled location.

In one embodiment, a densification method for pinpointing and quantifying maximal exposures to a population of individuals across a geographic region to environmental hazards is determined. In one embodiment, the hazards include radiation, pollution and communicable infectious agents. At one step, locations on the geographic map with the highest levels of uncertainty in radiation dose estimates is determined. At another step, additional environmental hazard assays at or close to those locations are sampled and their respective exposures are determined. At yet another step, a geographic map of the distribution of physical radiation exposures of all individuals proximate to the radiation source is recomputed using geostatistical methods that spatially infer radiation exposure contours from the measured radiation levels and the location of each sampled location, including the additional measurements.

At yet another step, a new set data on the quantifying exposures to a population of individuals across a geographic region to environmental hazards are obtained sequentially. At yet another step, locations on the geographic map with the highest levels of uncertainty in radiation dose estimates is determined. At yet another step, coincident between any of the location obtained by densification. At yet another step, the coincident location is assigned as the emerging hotspots for increased exposure to environmental hazards.

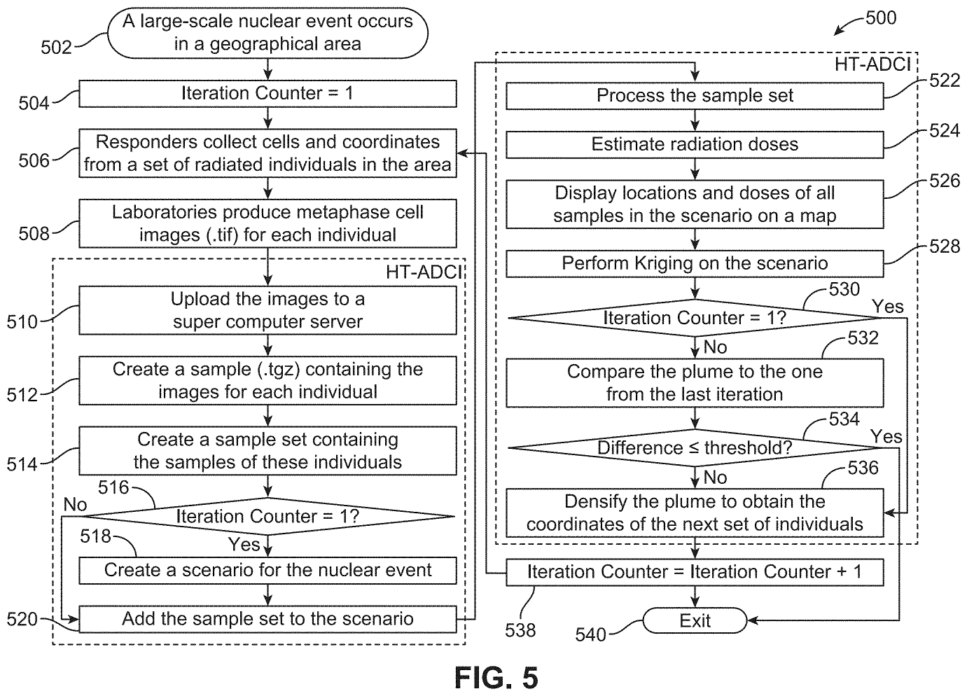

FIG. 5 exemplarily illustrates a flowchart of a method 500 for quantifying individual biological exposures to ionizing radiation in a population of individuals, according to an embodiment of the present invention. At step 502, consider a large-scale nuclear event occurs in a geographical area. At step, iteration counter=1. At step 506, responders collect cells and coordinates from a set of radiated individuals in the area. At step 508, laboratories produce metaphase cell images (.tif) for each individual. At step 510, the images are uploaded to a computer server. At step 512, a sample (.tgz) containing the images for each individual is created. At step 514, a sample set containing the samples of these individuals are created. At step 516, the method checks if the iteration counter=1. If the step 516 is satisfied, the method continues to step 518 and if the step 516 is not satisfied, the method continues to step 520. At step 518, a scenario for nuclear event is created. At step 520, the sample set is added to the scenario. At step 522, the sample set is processed. At step 524, the radiation doses are estimated. At step 526, locations and doses of all samples are displayed in the scenario on a map. At step 528, kriging is performed on the scenario. At step 530, check iteration counter=1. If step 530 is satisfied the method continues at step 536. If step 530 is not satisfied the method continues at step 532. At step 534, the plume is compared to the one from the last iteration. At step 534, the method checks if the difference in the compared plumes is lesser or equals to the threshold. At step 536, the plume is densified to obtain the coordinates of the next set of individuals. At step 538, check if iteration counter=iteration counter+1. At step 540, the method ends.

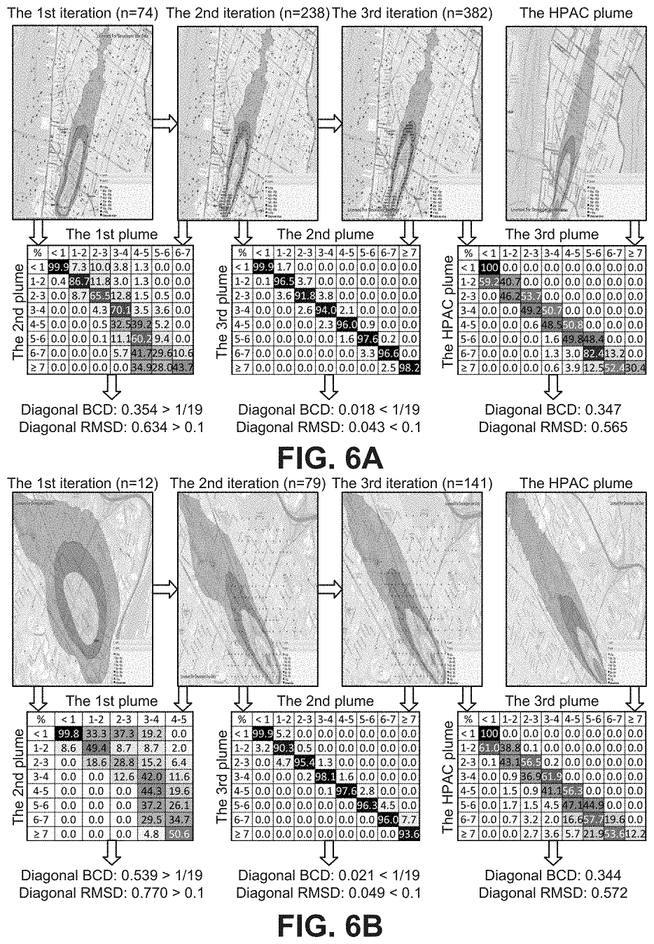

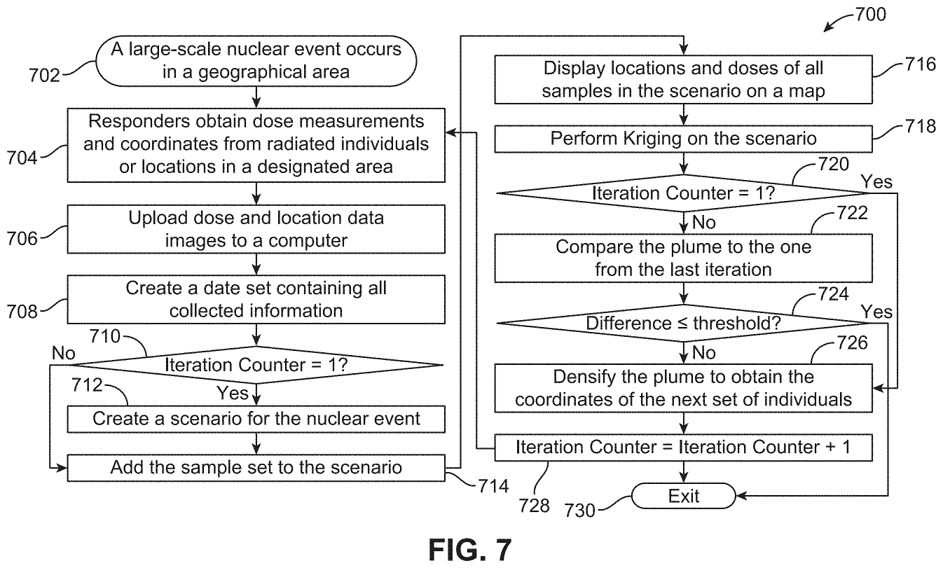

FIG. 6A and FIG. 6B exemplarily illustrates predicted radiation levels based on the distribution of dose estimated patients around the prediction location, according to an embodiment of the present invention. FIG. 7 exemplarily illustrates a flowchart of a method 700 for quantifying individual biological exposures to ionizing radiation in a population of individuals, according to another embodiment of the present invention. At step 702, a large-scale nuclear event occurs in a geographical area. At step 704, responders obtain dose measurements and coordinates from radiated individuals or locations in a designated area. At step 706, dose and location data of images are uploaded to a computer. At step 708, a date set containing all collected information is created. At step 710, check if iteration counter=1. If step 710 is satisfied the method continues to step 712. If step 710 is not satisfied the method continues to step 714. At step 712, a scenario for the nuclear event is created. At step 714, the sample set is added to the scenario. At step, the locations and doses of all samples in the scenario on a map is displayed. At step, kriging is performed on the scenario. At step 720, check if iteration counter=1. If step 720 is satisfied the method continues to step 726. If step 720 is not satisfied the method continues to step 722. At step 722, the plume is compared to the one from the last iteration. At step 724, the method checks if the difference in the compared plumes is lesser or equals to the threshold. At step 726, the plume is densified to obtain the coordinates of the next set of individuals. At step 728, check if iteration counter=iteration counter+1. At step 730, the method ends.

Further, comparison and advantages of the present invention over the existing method is disclosed as follows. Existing methods discloses about computing maps using infected individuals, where the municipalities of different sizes and populations are recorded as block support data. Instead of municipality the present invention uses county subdivisions. Block data refers to a non-point support data, which is generally referred to a volume but in this case of aggregated health data it refers to an area. This means the resulting high-resolution risk maps are point support based. In this regards the present invention and the existing method have similar objectives. However, the present invention randomly allocates cases within a county subdivision or “block” (and perform multiple replicates or simulations to verify results). Multiple replicates are typically analyzed (in which the distributions of cases are randomly generated at different locations within each county subdivision in each replicate). A minimum of 2 replicates are analyzed, but usually more. The reference for this is our recent PLOS ONE paper which is cited herein. This is done to mitigate against biased distributions of cases that can influence the outcome of spatial interpolation. In the existing method, a kriging technique that accounts simultaneously for point and block data is performed, where the block data, Bv(uα) Z(u′) are defined as the spatial linear average of point values Z(u′) within the block volume which in this particular case simplifies to an area.

The present invention combines spatial averages of point values within a block volume, and assigns the number of positive individuals within the county subdivision or “block”, to different random map locations to each individual based on population census of the subdivision. The present invention repeats this process for different sets of random locations several times. Each time, kriging is performed on the results. According to the present invention, testing six different known kriging methods and found the Empirical Bayesian Kriging provided the most accurate locations for sampling, i.e. hotspot locations, based on the known radiation exposures. An existing method of defining infection rates using kriging does not use densification to find new locations for sampling (which are similar to hotspot locations using the instant method). See, Azevedo, L., Pereira, M. J., Ribeiro, M. C. et al. Geostatistical COVID-19 infection risk maps for Portugal. Int J Health Geogr 19, 25 (2020). https://doi.org/10.1186/s12942-020-00221-5.) Further densification identifies the locations in which the spatial autocorrelations between neighbouring counties or subdivisions is lowest, with the highest variance. Instead existing method defines locations with highest infection risk (conceptually similar to hotspot). These authors apply population census information at this latter stage, whereas in the present invention the population census information is introduced at a later stage of the analysis as a corrective procedure to weight the rates of infection in municipalities based on their population sizes after the kriging step is performed (municipalities with large populations should have greater weighting in the experimental variogram calculation”). By contrast, overall population counts in a geographic region are incorporated by allocation of census data preceding kriging in the claimed invention.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

This disclosure uses terms such as “excessive counts” or “excessive infection counts”, e.g., “hotspots wherein said local counties exhibit excessive infection counts”. Other relative terms and contexts include “high and low”. Examples include “distributions of high and low infection counts”, “selecting interpolated locations with high variance, “pinpointing the locations of emerging hotspots at higher spatial resolution”, “highest levels of uncertainty”, “higher spatial resolution”, etc. The discussion below describes how the disclosed method establishes boundaries for these relative terms.

For the area to area asymmetry and cluster analysis (high versus low, clusters of high values or clusters of low values), we have used the Anselin local Moran's I index. The local Moran's I index (I) is a relative measure and can only be interpreted within the context of its computed z-score or p-value. The z-scores and p-values reported in the output feature class are uncorrected for multiple testing or spatial dependency. The cluster/outlier type (COType) field distinguishes between a statistically significant cluster of high values (HH), cluster of low values (LL), outlier in which a high value is surrounded primarily by low values (HL), and outlier in which a low value is surrounded primarily by high values (LH). Statistical significance can be set at a predetermined 95 percent confidence level, or at other selected levels. When no FDR correction is applied, features with p-values smaller than 0.05 are considered statistically significant. The FDR correction reduces this p-value threshold from 0.05 to a value that better reflects the 95 percent confidence level given multiple testing.

For the area to area Getis-Ord Gi* statistical hotspot analysis, it creates statistically significant spatial clusters of high values (hot spots) and low values (cold spots). It outputs parametric z-score, p-value, and confidence level bin field (Gi_Bin) for each type of environmental value that is analyzed. The z-scores and p-values are measures of statistical significance that tell you whether or not to reject the null hypothesis, feature by feature. In effect, they indicate whether the observed spatial clustering of high or low values is more pronounced than one would expect in a random distribution of those same values. The z-score and p-value fields do not reflect any type of FDR (False Discovery Rate) correction. We have performed these analysis both using this correction and without it. It corrects for multiple testing, i.e comparing the sample hypothesized hotspot location with multiple candidate neighbors. The Getis-Ord Gi* statistic identifies statistically significant hot and cold spots regardless of whether or not the FDR correction is applied. Features in the +/−3 bins reflect statistical significance with a 99 percent confidence level; features in the +/−2 bins reflect a 95 percent confidence level; features in the +/−1 bins reflect a 90 percent confidence level; and the clustering for features in bin 0 is not statistically significant. Without FDR correction, statistical significance is based on the p-value and z-score fields. When the False Discovery Rate (FDR) Correction is applied, the critical p-values determining confidence levels are reduced to account for multiple testing and spatial dependence. The z-score is based on the randomization null hypothesis computation. A high z-score and small p-value for a feature indicate a spatial clustering of high values. A low negative z-score and small p-value indicate a spatial clustering of low values. The higher (or lower) the z-score, the more intense the clustering. A z-score near zero indicates no apparent spatial clustering.

Regarding kriging: Kriging is an advanced geostatistical procedure that generates an estimated surface from a scattered set of points with z-scores. It assumes that values of points close to sampled points are more likely to be similar than those that are farther apart. This is the basis of interpolation of values at locations between known locations of environmental hazards. We have discussed the interpretation of z scores above, and they are well known in the art of statistics. The z scores can be used to derive the variance of spatial autocorrelation across the map. Densification selects points with the lowest (trending towards zero) Z scores on the map, ie. the lowest correlation average (eg. Z>=2) across a geographic set of locations are more likely to be hotspots (in the preceding example, equivalent to 90% confidence), whereas Z<=−2 tend to be cold spots. Thus, the parameters employed for kriging determine the equivalent confidence level.

Therefore, the selected confidence levels used in performing the method will effectively determine “high” and “low” values and further that once emerging hotspots are located by this procedure they would then be considered to exhibit, by definition, “excessive infection counts.” Thus, confidence level values can be selected thereby identifying high and low values, and counties with excessive infection counts, which emerge as the results of kriging and densification operations.

What is claimed is:

1. A method for identifying and quantifying populations exposed to environmental hazards across a geographic region, comprising:
a) inputting data into a computer having a processor, said data related to the source of the hazard, wherein the hazard is a communicable infectious agent, radiation, or pollution, wherein the data includes location of individuals infected by the infectious agents, number of infected individuals and location of the infected individuals or the radiation dose and locations of the radiation exposed individuals, or the concentrations of chemical pollutant exposures and locations of individuals in contact with chemical pollutants, and wherein the hazards include radiation, pollution and communicable infectious agents;
b) assaying a fraction of infected individuals of a local population and adjacent locations of the infected individuals;
c) increasing the spatial resolution of the assay by assigning infected cases in each county to subdivisions weighted by population census and performing spatial interpolation to pinpoint potential local clusters of infected individuals, and
d) assigning spatially interpolated maxima on the geographic map.

2. The method of claim 1, wherein the spatially interpolated maxima of the hazard on the geographic map is confirmed by performing area-to-area comparisons using geostatistical tests that identify existing hotspots by identifying,
(i) symmetric and clustered distributions of high and low infection counts among groups of neighbouring counties, or by,
(ii) defining local counties as hotspots wherein said local counties exhibit excessive infection counts relative to neighbouring counties, wherein excessive counts characterized by spatial interpolation of infection counts of individuals.

3. A densification method for quantifying exposures to a population of individuals across a geographic region to environmental hazards, said hazards including radiation, pollution, and communicable infectious agents, said method comprising the following steps:
a) inputting into a computer, having a processor, the target location, direction and rate of dispersal of the source of the hazard;
b) assaying dose at the locations of a fraction of individuals of a local population which is suspected of having been exposed to radiation, said fraction comprising at least 0.01% of the local population count, relative to the location of the target of the event and adjacent locations dictated by the direction of travel of the hazard;
c) determining the exposure level measured by assaying the environmental hazard at these locations;
d) computing a geographic map of the distribution of exposures to the environmental hazard of all individuals proximate to the radiation source using geostatistical methods that spatially infer exposure contours from the level of the environmental hazard and each sampled location;
e) determining locations on the geographic map of step (d) with the highest levels of uncertainty in radiation dose estimates;
f) sampling with additional environmental hazard assays at or close to those locations and determining their respective exposures;
g) recomputing a geographic map of the distribution of physical radiation exposures of all individuals proximate to the radiation source using geostatistical methods that spatially infer radiation exposure contours from the measured radiation levels and the location of each sampled location, including the additional measurements obtained in step (g);
h) sequentially obtaining a new set data on the quantifying exposures to a population of individuals across a geographic region to environmental hazards, said hazards including radiation, pollution, and communicable infectious agents;
i) determining locations on the geographic map of step (g), with the highest levels of uncertainty in radiation dose estimates;
j) determining if any of the locations obtained by densification in step (e) and Step (g) are coincident, and
k) assigning the coincident locations obtained in step (j) to be emerging hot spots for increased exposure to environmental hazards.

4. A method of quantifying individual biological exposures to ionizing radiation in a population of individuals comprising,
a) inputting the target location, into a computer having a processor, wind direction and speed of the radiation source;
b) sampling a fraction of localized individuals of the local population that may have been exposed to the radiation, said fraction comprising at least 0.01% of the local population count at the target location and adjacent locations dictated by the wind direction;
c) determining the absorbed biological radiation exposure level of sampled individuals by biodosimetry, and
d) computing a geographic map of the biological distribution of radiation exposures of all individuals proximate to the radiation source using geostatistical methods that spatially infer radiation exposure contours from the absorbed radiation level and the location of each sampled individual.

5. The method of claim 4, utilizes at least one of the tests including cytogenetic, gene and protein expression and metabolomic signatures, and electron paramagnetic resonance biodosimetry.

6. The method of claim 4, wherein the radiation levels are inferred at least one of ordinary kriging, simple kriging, universal kriging, empirical Bayesian kriging or non-linear regression method.

7. The method of claim 4, wherein the radiation exposure level is measured by placing physical dosimeters at the locations of a fraction of individuals of a local population which is suspected of having been exposed to radiation.

8. The method of claim 4, wherein the radiation exposure level is measured by placing testing equipment at the locations of a fraction of individuals of a local population which is suspected of having been exposed to radiation, and wherein the testing equipment includes Geiger Mueller detectors with pancake probes, alpha radiation survey meters, dose rate meters, personal dosimeters, and portal monitors.

9. The method of claim 4, further comprises a step of: improving the quantification of individual biological exposures to ionizing radiation in a population of individuals comprising,
e) determining locations on the biodosimetry geographic map of claim 1 with the highest levels of uncertainty in radiation dose estimates;
f) sampling additional individuals at or close to those locations and determining their respective biological radiation exposure levels, and
g) recomputing a geographic map of the biological distribution of radiation exposures of all individuals proximate to the radiation source using geostatistical methods that spatially infer radiation exposure contours from the absorbed radiation level and the location of each sampled individual, including the additional individuals sampled in the above step (f).

10. A method for geostatistical analysis of infections of individuals with a communicable pathogen comprising,
retrieving, curating, and preparing county-level incidence data for geostatistical analysis, using geostatistical tests that identify existing hotspots by identifying,
(i) asymmetric and clustered distributions of high and low infection counts among groups of neighboring counties, or by
(ii) defining local counties as hotspots wherein said local counties exhibit excessive infection counts relative to neighboring counties, wherein excessive counts characterized by spatial interpolation of infection counts of individuals,
inferring locations of existing hotspots,
integrating data from consecutive dates at locations where existing hotspots have been inferred,
pinpointing the locations of emerging hotspots at higher spatial resolution by geostatistical interpolation, by reallocating cases of said infections across county subdivisions based on corresponding population census data,
identifying locations of said emerging hotspots of infected persons at sub-county resolution by
reallocating cases of across county subdivisions based on corresponding population census data,
selecting interpolated locations with high variance in interpolated incidence levels.
wherein said interpolated locations exhibit a loss of spatial autocorrelation due to the presence of said emerging hotspots against lower background levels in surrounding counties,

11. The method of claim 10 wherein the locations of emerging hotspots are confirmed by performing area-to-area comparisons using geostatistical tests that identify existing hotspots by identifying,
(i) symmetric and clustered distributions of high and low infection counts among groups of neighboring counties, or by
(ii) defining local counties as hotspots wherein said local counties exhibit excessive infection counts relative to neighboring counties, wherein excessive counts characterized by spatial interpolation of infection counts of individuals.

12. The method of claim 10 wherein the identified emerging hotspots represent spatially interpolated locations with maximum values in their respective areas.

13. The method of claim 10 further comprising,
developing a composite risk score that combines results of joinpoint regression modeling of temporal trends and boundary analysis.

14. The method of claim 10 wherein said geostatistical interpolation is performed using Empirical Bayesian Kriging.

15. The method of claim 11 wherein said geostatistical interpolation is performed using Empirical Bayesian Kriging.

16. The method of claim 12 further comprising,
confirming the locations of emerging hotspots by area-to-area geostatistical analyses using either Getis-Ord Gi* or Anselin Local Moran's I testing on the date of kriging, or by integrating the geostatistical tests with temporal analysis over a range of dates.

17. The method of claim 12 further comprising, wherein said geostatistical interpolation is performed using Empirical Bayesian Kriging.

18. The method of claim 17 further comprising,
confirming the locations of emerging hotspots by area-to-area geostatistical analyses using either Getis-Ord Gi* or Anselin Local Moran's I testing on the date of kriging, or by integrating the geostatistical tests with temporal analysis over a range of dates.

19. The method of claim 3 wherein the locations of obtained by densification are confirmed by performing area-to-area comparisons using geostatistical tests that identify existing hotspots by identifying,
(i) symmetric and clustered distributions of high and low infection counts among groups of neighboring counties, or by
(ii) defining local counties as hotspots wherein said local counties exhibit excessive infection counts relative to neighboring counties, wherein excessive counts characterized by spatial interpolation of infection counts of individuals.
